(19) United States
(12) Patent Application Publication
(10) Pub. No.: US 2004/0204606 A1
(43) Pub. Date: Oct. 14, 2004

(54) Polymer-bound catalyst for the enantioselective cleavage of pro-chiral anhydrides

(76) Inventors: Jens Woltinger, Hanau (DE); Hans-Peter Krimmer, Kirchweidach (DE); Dietmar Reichert, Eschau (DE); Juan Jose Almena Perea, Hanau (DE); Karlheinz Drauz, Freigericht (DE); Andreas Karau, Neustadt (DE)

Correspondence Address: Fitch Even, Tabin & Flannery, Suite 401L, 1801 K Street NW, Washington, DC, 20006-1201, US

(21) Appl. No.: 10/475,752

(22) PCT Filed: Mar. 20, 2002
(86) PCT No.: PCT/EP02/03050

(30) Foreign Application Priority Data
Apr. 26, 2001 (DE) 101 20 455.8
Feb. 27, 2002 (DE) 102 08 592.7

(51) Int. Cl. C07C 51/087

(52) U.S. Cl. 562/401

(57) ABSTRACT

The invention relates to the use of catalysts comprising the structures of general formulas (I), (II) or (III), for the asymmetric cleavage of anhydrides.

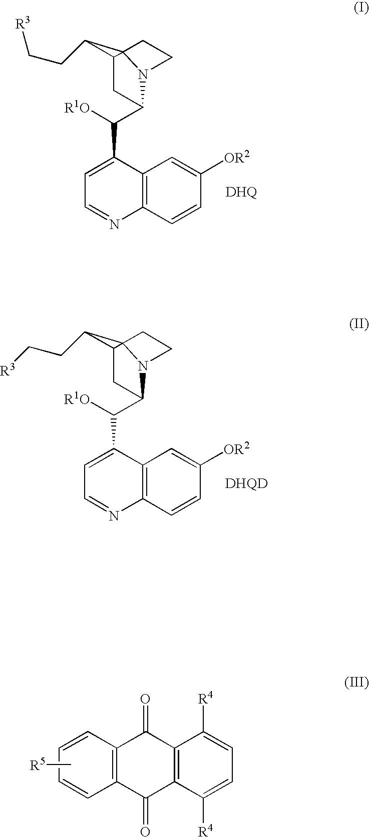

The present invention is directed to the use of optically active polymer-enlarged catalysts. In particular the invention relates to polymer-enlarged catalysts having one or more structures of the general formulae (I) or (II) as active unit resulting in the chiral induction

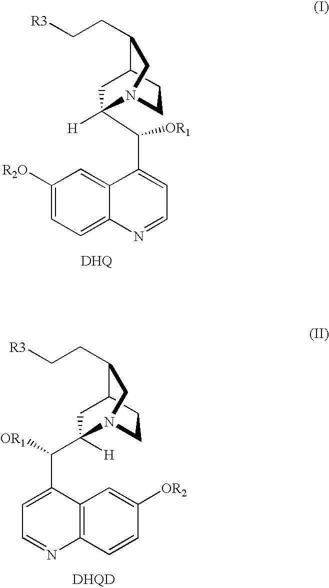

Homogeneously soluble polymer-enlarged-chiral catalysts are important auxiliary substances in the synthesis of enantiomer-enriched organic compounds particularly also on an industrial scale, and on account of their catalytic activity on the one hand and their ability to be recycled and reused on the other hand, are able to facilitate the preparation of the desired products in an extremely cost-effective manner. Furthermore, the reactions carried out with them do not have the phase changes of the substrate and product influencing the reaction that are inherent in the use of heterogeneously soluble polymer-enlarged catalysts. In addition there is a need for catalysts for use in the organic catalytic synthesis of chiral compounds.

Janda and Bolm et al. have reported on examples of quinine/quinidine ligands that have recently been synthesised (Chem. Commun. 1999, 1917-1924; Eur. J. Org. Chem. 1988, 21-27).

Homogeneously soluble polymer-enlarged quinine/quinidine ligands are already known in the field of the enantioselective dihydroxylation reaction according to Sharpless (J. Am. Chem. Soc. 1996, 118, 7632; Tetrahedron Lett. 1997, 38, 1527; Angew. Chem. Int. Ed. Engl. 1997, 36, 773).

It is furthermore known to use quinine/quinidine ligands as catalysts in the enantioselective cleavage of pro-chiral anhydrides (Deng et al., J. Am. chem. Soc. 2000,. 9542; Bolm et al., Synlett, 1999, 2, 195-196).

The object of the present invention was accordingly to discover further uses of homogeneously soluble polymer-enlarged catalysts of the quinine/quinidine type for the asymmetric synthesis of organic compounds.

This object is achieved by the use of the catalysts having the features of claim 1. Claims 2 to 6 relate to specific modifications of the protected use according to the invention.

Accordingly, if in a process for the asymmetric cleavage of pro-chiral anhydrides there are used optically active homogeneously soluble polymer-enlarged catalysts having as active unit causing the chiral induction, one or more structures of the general formulae (I), (II) or (III)

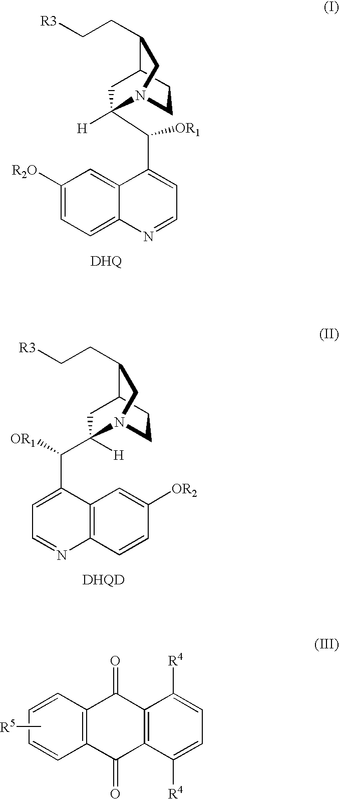

wherein

R¹, R² independently of one another denote H, (C₁-C₈)-alkyl, (C₁-C₈)-acyl, (C₃-C₈)-cycloalkyl, (C₆-C₁₈)-aryl, (C₇-C₁₉)-aralkyl, (C₃-C ₁₈)-heteroaryl, (C₄-₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃- (C₃-C₁₈) -heteroaryl,

R³ is H,

or R¹, R² or R³ is the bonding to the polymer enlargement,

R⁴ denotes DHQ (I) or DHQD (II) and R⁵ is H or the bonding to a polymer,

then the desired optically enriched compounds are obtained in excellent yields and with high enantiomer excesses. At the same time, due to the polymer bonding, the catalysts can very easily be separated from the low molecular weight compounds, for example by filtration, and are therefore accessible to the extremely simple but no less advantageous desired recycling according to the invention.

Polymer Enlargement:

The polymer enlargement may be freely chosen within the scope of the invention. The enlargement is restricted on the one hand by considerations of practicability and cost, and on the other hand by technical boundary conditions (retention capacity, solubility, etc.). Some polymer enlargements for catalysts are known from the prior art (Reetz et al., Angew. Chem. 1997, 109, 1559f.; Seebach et al., Helv. Chim. Acta 1996, 79, 1710f.; Kragl et al., Angew. Chem. 1996, 108, 684f.; Schurig et al., Chem. Ber./Recueil 1997, 130, 879f.; Bolm et al., Angew. Chem. 1997,:109, 773f.; Bolm et al., Eur. J. Org. Chem. 1998, 21f.; Baystone et al. in Speciality Chemicals 224f.; Salvadori et al., Tetrahedron: Asymmetry 1998, 9, 1479; Wandrey et al., Tetrahedron: Asymmetry 1997, 8, 1529f.; ibid. 1997, 8, 1975f.; Togni et al., J. Am. Chem. Soc. 1998, 120, 10274f., Salvadori et. al., Tetrahedron Lett. 1996, 37, 3375f; WO 98/22415;especially DE 19910691.6; Janda et al., J. Am. Chem. Soc. 1998, 120, 9481f.; Andersson et al., Chem. Commun. 1996,. 1135f.; Janda et al., Soluble Polymers 1999, 1, 1; Janda et al., Chem. Rev. 1997, 97, 489; Geckler et al., Adv. Polym. Sci. 1995, 121, 31; White et al. al. in “The Chemistry of Organic Silicon Compounds”, Wiley, Chichester, 1989, 1289; Schuberth et al., Macromol. Rapid Commun. 1998, 19, 309; Sharma et al., Synthesis 1997, 1217; “Functional Polymers”, Ed.: R. Arshady, ASC, Washington, 1996; “Praktikum der Makromolekularen Stoffe”, D. Braun et al., VCH-Wiley, Weinheim.1999).

Furthermore, the polymer enlargement is preferably effected by polyacrylates, polyacrylamides, polyvinylpyrrolidinones, polysiloxanes, polybutadienes, polyisoprenes, polyalkanes, polystyrenes, polyoxazolines or polyethers, or mixtures thereof. In a most particularly preferred modification polystyrenes are used for effecting the polymer enlargement.

Linkers:

A linker may be incorporated between the actual active unit and the polymer enlargement. The linker serves to create an interspacing between the active unit and polymer in order to lessen or exclude interactions that are disadvantageous for the reaction. The linkers may in principle be freely chosen by the person skilled in the art. They should be chosen according to how well they can be coupled on the one hand to the polymer/monomer, and on the other hand to the active centre. Suitable linkers may be found inter alia from the literature sources cited above under the heading “polymer enlargement”. Within the scope of the invention these active units of the formulae (I) to (III) advantageously, i.e. directly or preferably, bonded to the polymer enlargement by means of a linker selected from the following group

| | | |
|---|---|---|
| a) | —Si(R₂)— | |
| b) | —(SiR₂—O)ₙ— | n = 1-10000 |
| C) | —(CHR—CHR—O)ₙ— | n = 1-10000 |
| d) | —(X)ₙ— | n = 1-20 |
| e) | Z—(X)ₙ— | n = 0-20 |
| f) | —(X)ₙ—W | n = 0-20 |
| g) | Z—(X)ₙ—W | n = 0-20 |

wherein

R denotes H, (C₁-C₈)-alkyl, (C₆-C₁₈)-aryl, (C₇-C₁₉)-aralkyl, ((C₁-C₈)-alkyl)₁₋₃-(C₆-C₁₈)-aryl,

X denotes (C₆-C₁₈)-arylene, (C₁-C₈)-alkylene, (C₁-C₈)-alkenylene, ((C₁-C₈)-alkyl)₁₋₃-(C₆-C₁₈)-arylene, (C₇-C₁₉)-aralkylene,

Z, W denote independently of one another —C(═O)O—, —C(═O)NH—, —C(═O)—, NR, O, CHR, CH₂, C═S, S, PR.

Further preferred compounds that may be used as linkers are shown hereinafter:

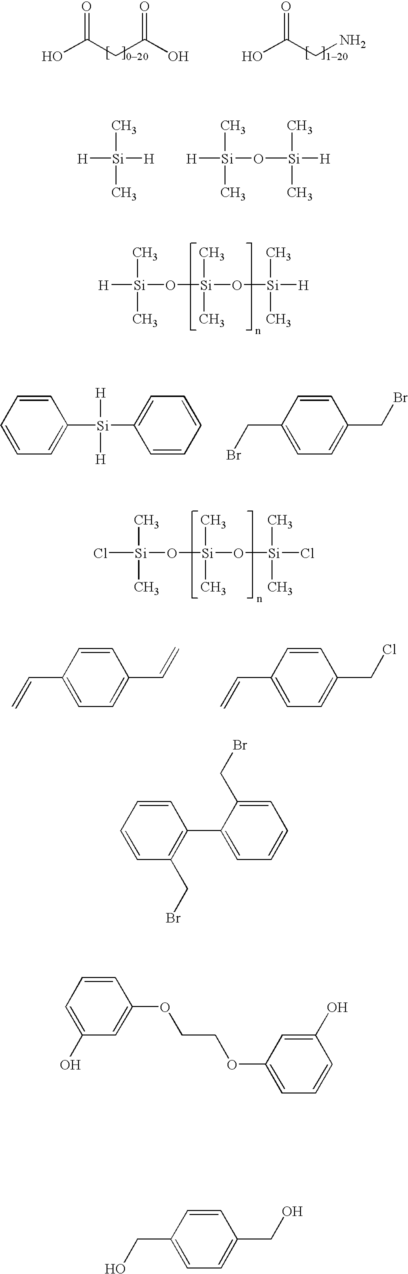

Most particularly preferred however are linkers such as for example 1,4′-biphenyl, 1,2-ethylene, 1,3-propylene, PEG-(2-10), α,ω-siloxanylene or 1,4-phenylene as well as α,ω-1,4-bisethylenebenzene or linkers that are obtainable starting from siloxanes of the general formula IV

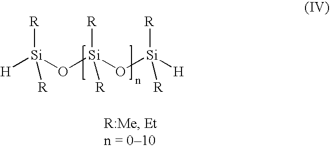

These can readily bond under hydrosilylation conditions (review of the hydrosilylation reaction of Ojima in The Chemistry of Organic Silicon Compounds, 1989, John Wiley & Sons Ltd., 1480-1526) to double bonds possibly present in the polymers and to suitable functional groups of the active centres.

The size of the polymer enlargement should be dimensioned so that the catalyst dissolves in the solvent to be used, i.e. so that the reaction can be carried out in the homogeneous phase. The catalyst that is used is therefore preferably an homogeneously soluble catalyst. In this way negative effects that occur due to the phase changes of the substrates and products that are otherwise necessary when using heterogeneous catalysts can be avoided. The polymer enlarged catalysts may have a mean molecular weight in the range from 1000 to 1,000,000 g/mole, preferably 5000 to 500,000 g/mole and particularly preferably 5000 to 300,000 g/mole.

Within the scope of the invention and on the basis of the specialist knowledge of a person skilled in the art the aforementioned constituents of the polymer-enlarged catalysts (I), to (III) (polymer, linker, active centre/unit) can be combined arbitrarily having regard to optimum reaction conditions.

Combination of Polymer Enlargement and Linker/Active Unit:

In principle there are two possible ways by which the linker/active unit can be involved in the polymer enlargement:

a) the active unit resulting in the chiral induction is bound via a connected linker or directly to a monomer and this is copolymerised with further unmodified monomers, or

b) the active unit-resulting in the chiral induction is bound via a linker or directly to the finished polymer.

Optionally polymers may be produced according to a) or b) and then block-copolymerised with other polymers that likewise have the active units resulting in the chiral induction or do not have such units.

Furthermore, it is true in principle as regards the number of linkers/active units per monomer in the polymer that as, many such catalytically active units as possible should be available on a polymer so that the conversion per polymer is thereby increased. On the other hand the units should however adopt such an interspacing that a mutual negative influencing of the reactivity (TOF (turnover frequency), selectivity) is minimised or indeed is prevented. Preferably the mutual interspacing of the linkers/active centres in the polymer should therefore be in the range from 1 to 200 monomer units, preferably 5 to 25 monomer units.

In an advantageous modification those sites in the polymer or monomer to be polymerised are involved in the bonding of the linker/active unit that can easily be functionalised or that already enable an existing functionality to be used for the bonding. Thus, heteroatoms or unsaturated carbon atoms are ideally suitable for effecting the bonding.

For example, in the case of styrene/polystyrene the existing aromatic compounds may be used as binding sites to the linkers/active centres. Functional groups may readily be coupled by normal aromatic chemistry techniques to these aromatic compounds, preferably in the 3-, 4- or 5-position, particularly preferably in the 4-position. It is however also advantageous to mix for example already functionalised monomer-with the mixture to be polymerised, and after the polymerisation to bind the linker to the functional groups already present in the polystyrene. p-hydroxystyrene, p-chloromethylstyrene or p-aminostyrene derivatives are for example suitable for this purpose.

In the case of polyacrylates an acidic group or ester group is in each case present in the monomer constituent, to which the linker or the active unit can be bound, preferably via an ester bond or amide bond, either before or after the polymerisation.

Polysiloxanes as polymer enlargement agent are preferably uniformly synthesised so that, in addition to dimethylsilane units, hydroxymethylsilane units are also present. The linkers/active units may then be coupled to these sites via an hydrosilylation reaction. Preferably these can bond under hydrosilylation conditions (review of the hydrosilylation reaction of Olima in The Chemistry of Organic Silicon Compounds, 1989, John Wiley & Sons Ltd., 1480-1526) to the envisaged functional groups in the polymer.

Suitable polysiloxanes modified in this way are known in the literature (“Siloxane polymers and copolymers”, White et al., in Ed. S. Patai “The Chemistry of Organic Silicon Compounds”, Wiley, Chichester, 1989, 46, 2954; C. Wandrey et al. TH: Asymmetry 1997, 8, 1975).

Combination of Linker and Active Unit:

The above description regarding the binding of polymer to linker/active unit applies equally as regards the binding of the active centre (active unit) to the linker.

Thus, the binding of the linker to the active units may preferably take place via heteroatoms or specific functional groups such as C═O, CH₂,O, N, S, P, Si, B, wherein preferably ether/thioether bonds, amine bonds or amide bonds are coupled, or esterifications, alkylations, silylations as well as additions to double bonds are carried out.

Particularly preferred are those bonding possibilities that have already been described in the prior art for the polymer enlargement of the monomeric active units (WO98/35927; Chem. Commun. 1999, 1917; Angew. Chem. 1997, 16, 1835;. J. Am. Chem. Soc. 1996, 118, 7632; Tetrahedron Lett. 1997, 38, 1527; Eur. J. Org. Chem. 1998, 21; Angew. Chem. 1997, 109, 773; Chem. Commun. 1997, 2353; Tetrahedron: Asymmetry-1995, 6, 2687; ibid. 1993, 4, 2351; Tetrahedron Lett. 1995, 36, 1549; Synlett 1999, 8, 1181; Tetrahedron: Asymmetry 1996, 7, 645; Tetrahedron Lett. 1992, 33, 5453; ibid. 1994, 35, 6559; Tetrahedron 1994, 50, 11321; Chirality 1999, 11:, 745; Tetrahedron Lett. 1991, 32, 5175; Tetrahedron Lett. 1990, 31, 3003; Chem. Commun. 1998, 2435; Tetrahedron Lett. 1997, 38, 2577).

The preparation of a polymer-enlarged catalyst for the purpose according to the invention may in principle also be carried out according to the procedure described in DE10029600.

The active units are preferably coupled via the following groups of compounds to the polymers/linkers.

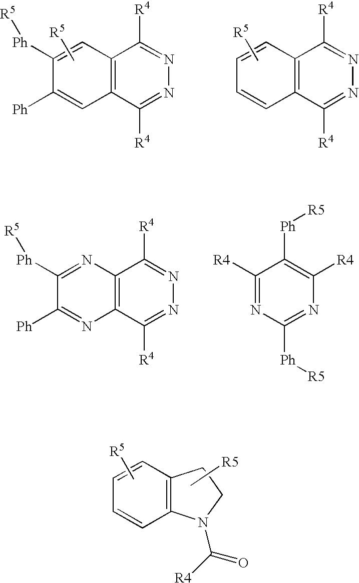

The definitions given hereinbefore apply to the radicals R⁴ and R⁵. The illustrated structures may, conveniently be coupled to the polymer/linker via the radicals R⁵, wherein at least one of the specified bonding possibilities per compound should be realised.

The catalysts under consideration are particularly preferably used for the asymmetric cleavage of pro-chiral anhydrides, the process being carried out in a membrane reactor. The continuous procedure that is possible in addition to the batch and semi-continuous procedures in this apparatus may be carried out as desired in crossflow filtration mode (FIG. 2) or as dead-end filtration (FIG. 1).

Both process variants are in principle described in the prior art (Engineering Processes for Bioseparations, Ed.: L. R. Weatherley, Heinemann, 1994 135-165; Wandrey et al., Tetrahedron Asymmetry 1999, 10, 923-928).

It is particularly preferred to employ the present invention in situations where the reaction is carried out in repetitive batch mode and the catalyst in the membrane reactor is washed between the reactions. Repetitive batch means that the reaction is carried out several times in succession, wherein at the start the reactants are added to the reactor and after completion of the reaction the products are removed before the reactor is recharged. The catalyst remaining in the reactor is in this connection washed in each case before the reactor is charged with the reactants. The object of the washing is to remove from the reactor reaction residues such as product, educt and the like that are still adhering to the reactor, in order to ensure in each case uniformly satisfactory starting conditions. It has been found that the products are to some extent bound by the catalyst (e.g. acid-base pair). The washing may therefore preferably also serve and may be executed to ensure that no product molecules remain chemically or physically bound in the reactor. On the other hand it is however also possible to operate this reaction of the anhydrides with the catalyst under conditions that help to suppress a reaction of the product with the catalyst. Such conditions would for example involve adding further bases to the reaction in order to reverse the acid-base reaction of the products with the catalyst.

In order for a catalyst to be suitable for use in a membrane reactor, it must satisfy a very wide range of criteria. Thus, on the one hand care should be taken to ensure that an appropriately high retention capability for the polymer-enlarged catalyst exists so that it has a satisfactory activity in the reactor over a desired timescale without the catalyst having to be continuously replenished, which is disadvantageous as regards operational economy (DE19910691). Furthermore the catalyst that is used should have an appropriate TOF in order to be able to convert the substrate into the product in economically favourable timescales. The catalysts of the quinine/quinidine type known from the prior art with reaction velocities in the timescale of 2 hours and ee values of 85% have been optimised for use in the membrane reactor so as to have reaction velocities of 15 minutes and >90%ee. To this end the catalyst has to be used in an up to tenfold excess compared to the anhydride. By virtue the concept of in situ recycling of the catalyst by means of the membrane reactor an economic operation is possible despite the high catalyst charge.

The production of a polymer-enlarged (DHQ)₂AQN catalyst may take place as follows.

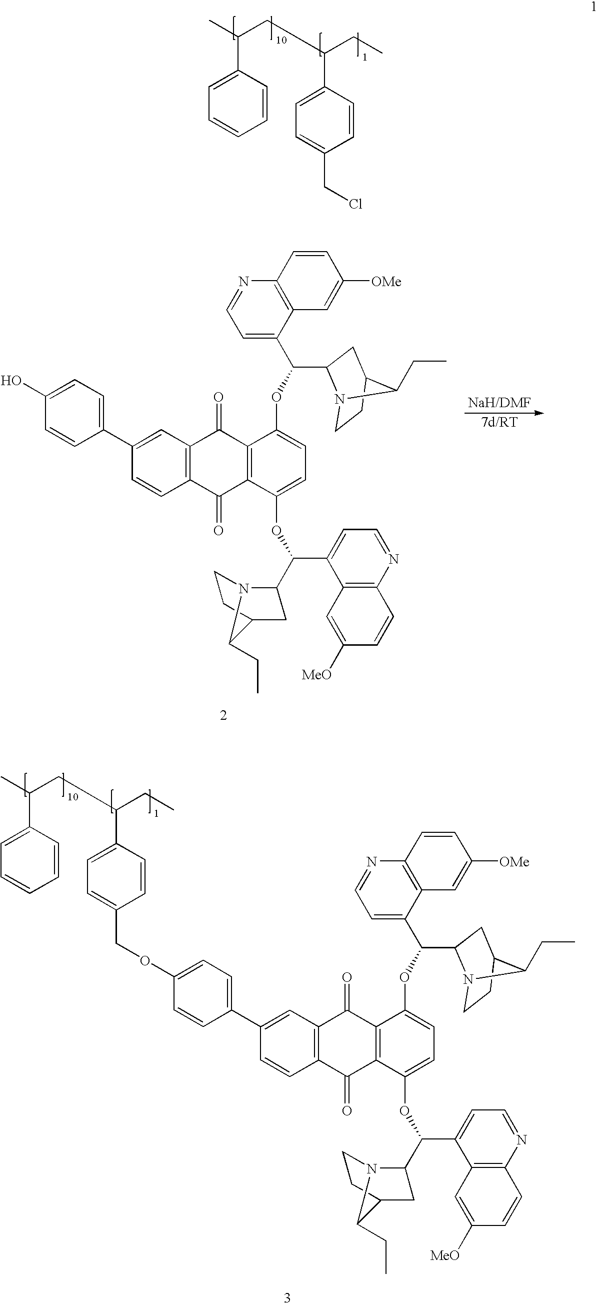

With this catalyst the following reaction was carried out in repetitive batch mode in the membrane reactor.

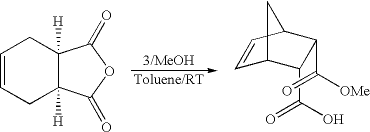

Conditions: repetitive batch in the dead-end apparatus (FIG. 1) with 10 ml toluene, 0.25 mmole anhydride, 2.5 mmole methanol per batch and a total amount of 0.25 mmole catalyst

The result is illustrated in FIG. 3. It can be seen that, as regards the conversion, an almost quantitative yield is achieved. The ee value however falls dramatically after a short time. The ee value can be maintained constant at ca. 60% with an equally good conversion by washing the catalyst once or several times in the membrane reactor between the individual runs, e.g. with a solution of toluene and methanol (10 equivs. methanol referred to the anhydride employed), followed by washing once or several times with pure toluene. An NMR protocol of the catalyst used then showed however that 2 equivs. of product were present per AQN unit, from which it can be concluded that the product (acid) appears to be bound to the catalyst (base). This could be the reason why the ee value does not have the quality exhibited by the monomeric ligand, since the monomeric ligand is used only once and therefore a formation of the acid-base pair cannot influence the selectivity. This is also evident when using the molecular weight-enlarged catalyst 3, since here too a selectivity comparable to the monomeric catalyst is achieved in the first batch. Only further uses of the catalyst lead to a reduction of the selectivity due to the formation of the product-catalyst pair. This complex can be dissociated by suitable protocols for the washing of the catalyst between the batch tests in a repetitive batch procedure, whereby the selectivity can be raised. (FIG. 3) The principle of carrying out this reaction in a membrane reactor has however been impressively demonstrated.

Mixtures of polymer-enlarged polymers are understood within the scope of the invention to mean that individual polymers of different origin are polymerised together to form block polymers. Random mixtures of the monomers in the polymer are also possible.

Polymer enlargement is understood within the scope of the invention to mean that one or more active units resulting in the chiral induction are copolymerised in a form suitable for this purpose with further monomers, or that this unit/these units are coupled to a polymer already present according to methods known to the person skilled in the art. Types of units suitable for the copolymerisation are well known to the person skilled in the art and can be freely chosen by the latter. The preferred procedure is, depending on the nature of the copolymerisation, to derivatise the molecule in question with groups capable of undergoing copolymerisation, for example in the case of copolymerisation with (meth)acrylates by coupling to acrylate molecules.

As (C₁-C₈)-alkyl there may be used methyl, ethyl, n-propyl, isopropyl, n-butyl, isobutyl, sec-butyl, tert-butyl, pentyl, hexyl, heptyl or octyl, as well as all bond isomers.

A (C₆-C₁₈)-aryl radical is understood to denote an aromatic radical with 6 to 18 C atoms. In particular this includes compounds such as phenyl, naphthyl, anthryl, phenanthryl and biphenyl radicals. These may be singly or multiply substituted with (C₁-C₈)-alkoxy, (C₁-C₈) -haloalkyl, OH, Cl, NH₂ or NO₂. Also the radical may have one or more heteroatoms such as N, O, S.

(C₁-C₈)-alkoxy is a (C₁-C₈)-alkyl radical bonded via an oxygen atom to the molecule in question.

A (C₇-C₁₉)-aralkyl radical is a (C₆-C₁₈)-aryl radical bonded via a (C₁-C₈) -alkyl radical to the molecule.

The term acrylate is understood within the scope of the invention to include also the term methacrylate.

(C₁-C₈)-haloalkyl denotes a (C₁-C₈)-alkyl radical substituted with one or more halogen atoms. Suitable halogen atoms are in particular chlorine and fluorine.

A (C₃-C₁₈)-heteroaryl radical denotes within the scope of the invention a 5-membered, 6-membered or 7-membered aromatic ring system containing 3 to 18 C atoms that has heteroatoms such as for example nitrogen, oxygen or sulfur in the ring. Heteroaromatic radicals are in particular radicals such as 1-, 2-, 3-furyl, such as 1-, 2-, 3-pyrrolyl, 1-, 2-, 3-thienyl 2-, 3-, 4-pyridyl, 2-, 3-, 4-, 5-, 6-,7-7indolyl, 3-, 4-, 5-pyrazolyl,2-, 4-, 5-imidazolyl, acridinyl, quinolinyl, phenanthridinyl or 2-, 4-, 5-, 6-pyrimidinyl. This may be singly or multiply substituted with (C₁-C₈)-alkoxy, (C₁-C₈)-haloalkyl, OH, halogen, NH₂, NO₂, SH, S—(C₁-C₈)-alkyl.

A (C₄-C₁₉)-heteroaralkyl is understood to denote an heteroaromatic system corresponding to the (C₇-C₁₉)-aralkyl radical.

The term (C₁-C₈)-alkylene chain is understood to denote a (C₁-C₈)-alkyl radical that is bonded via two different C atoms to the relevant molecule. This may be singly or multiply substituted with (C₁-C₈) -alkoxy, (C₁-C₈)-haloalkyl, OH, halogen, NH₂, NO₂, SH, S—(C₁-C₈)-alkyl.

(C₃-C₈)-cycloalkyl is understood to denote cyclopropyl, cyclobutyl, cyclopentyl, cyclohexyl, cycloheptyl or cyclooctyl radicals.

Halogen is fluorine, chlorine, bromine or iodine.

Within the scope of the invention the term membrane reactor is understood to mean any reaction vessel in which the catalystis enclosed in a reactor, while low molecular weight substances can be added to the reactor or can leave the latter. In this connection the membrane may be integrated directly in the reaction space or may be incorporated outside in a separate filtration module, in which the reaction solution flows continuously or intermittently through the filtration module and the retentate is recycled to the reactor. Suitable embodiments are described inter alia in WO98/22415 and in Wandrey et al. in the 1998 Yearbook, Verfahrenstechnik und Chemieingenieurwesen, VDI p. 151ff.; Wandrey et al. in Applied Homogeneous Catalysis with Organometallic Compounds, Vol. 2, VCH 1996, p. 832ff.; Kragl et al., Angew. Chem. 1996, 6, 684f.

The illustrated chemical structures refer to all possible stereoisomers that can be obtained by altering the configuration of the individual chiral centres, axes or planes, i.e. all possible diastereomers, as well as all optical isomers covered by the latter (enantiomers). It should however be pointed out that, within a polymer-enlarged catalyst, all active units present should according to the invention have the same chirality.

### DESCRIPTIONS OF THE DRAWINGS

FIG. 1 shows a membrane reactor with dead-end filtration. The substrate 1 is transferred via a pump 2 to the reactor space 3, which has a membrane 5. The stirrer-operated reactor space includes, in addition to the solvent, the catalyst 4 the product 6 and unreacted substrate 1. Mainly low molecular weight product 6 is filtered off through the membrane 5.

FIG. 2 shows a membrane reactor with crossflow filtration. The substrate 7 is in this case transferred via the pump 8 to the stirred reactor space, which also contains solvent, catalyst 9 and product 14. By means of the pump 16 a solvent flow is established that passes through an optionally present heat exchanger 12 to the crossflow filtration cell 15. Here the low molecular weight product 14 is separated by the membrane 13. High molecular weight catalyst 9 is then recycled together with the solvent flow optionally through a heat exchanger 12 and optionally through the valve 11 to the reactor 10.

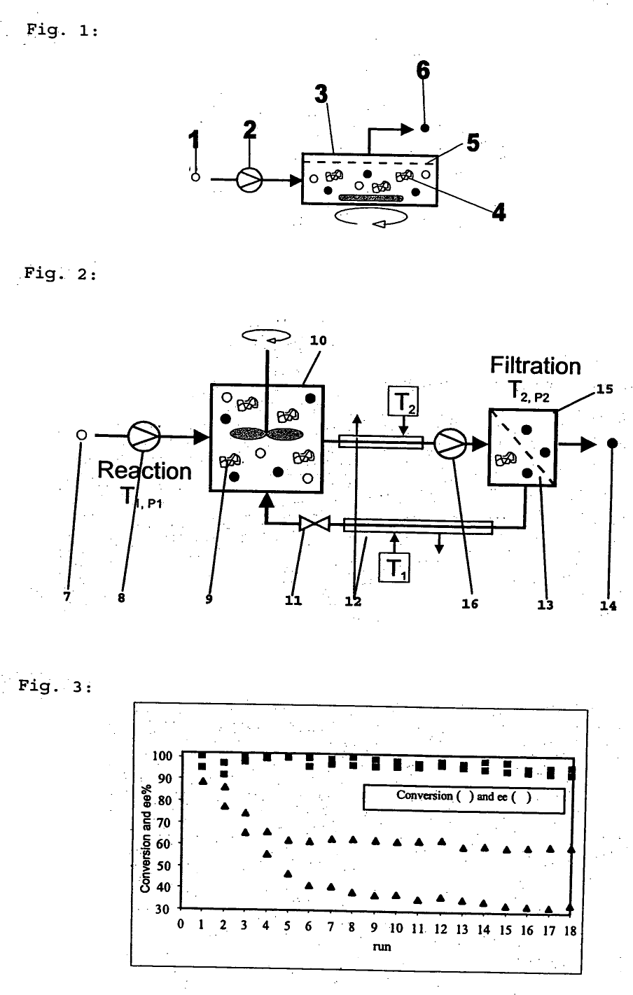

### EXAMPLES

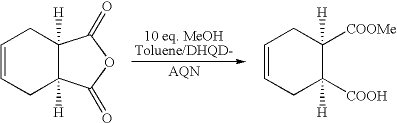

81.4 mg (0.5 mmole) of cis-4-cyclohexene-1,2-dicarboxylic anhydride (95%) and 449.6 mg of (DHQ)₂AQN catalyst (0.5 mmole) are suspended in 10 ml of toluene. A clear homogeneous solution was obtained by adding 63.3 μl of methanol (2.5 mmole). After 15 minutes no educt could be detected by means of HPLC. The product was formed with enantiomer excess of 90.4%.

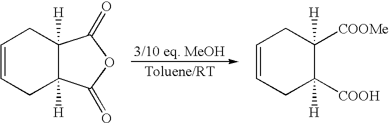

The reaction was carried out in 10 ml of toluene. 0.25 mmole of anhydride and 2.5 mmole of methanol were used. Catalyst 3 was employed as catalyst. After the end of the reaction the reaction solution was forced out from the reactor through a membrane. The molecular weight-enlarged catalyst 3 remained however behind the membrane. The next batch experiment was started by addition of the educts, which were dissolved in toluene.

1-9. (canceled)

10. In a process for the asymmetric cleavage of a pro-chiral anhydride, the improvement comprising catalyzing said cleavage with an optically active homogeneously soluble polymer-enlarged catalyst, wherein said catalyst comprises:
a) an active unit resulting in chiral induction, wherein said active unit has the structure of formula (I), (II) or (III): 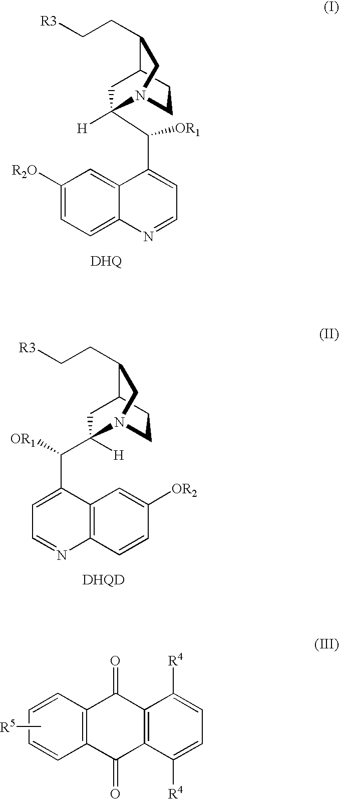
wherein:
R¹ and R² each independently are selected from: H, (C₁-C₈)-alkyl, (C₁-C₈)-acyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl,
R³ is H,
R⁴ is DHQ (I) or DHQD (II),
R⁵ is H,
or, any one or more of R¹, R², R³, R⁴, or R⁵, may, alternatively, be a bond to a polymer; and
b) at least one polymer, bound to said active unit by any one of R¹-R⁵.

11. The process of claim 1, wherein said active unit is bound to said polymer by a linker selected from the group consisting of:

| | | |
|---|---|---|
| a) | —Si(R₂)— | |
| b) | —(SiR₂—O)ₙ— | n = 1-10000 |
| c) | —(CHR—CHR—O)ₙ— | n = 1-10000 |
| d) | —(X)ₙ— | n = 1-20 |
| e) | Z—(X)ₙ— | n = 0-20 |
| f) | —(X)ₙ—W | n = 0-20 |
| g) | Z—(X)ₙ—W | n = 0-20 |

wherein
R denotes H, (C₁-C₈)-alkyl, (C₆-C₁₈)-aryl, (C₇-C₁₉)-aralkyl, ((C₁-C₈)-alkyl)₁₋₃-(C₆-C₁₈)-aryl,
X denotes (C₆-C₁₈)-arylene, (C₁-C₈)-alkylene, (C₁-C₈)-alkenylene, ((C₁-C₈)-alkyl)₁₋₃-(C₆-C₁₈)-arylene, (C₇-C₁₉)-aralkylene,
Z, W denote independently of one another —C(═O)O—, —C(═O)NH—, —C(═O)—, NR, O, CHR, CH₂, C═S, S, and PR.

12. The process of either claim 10 or 11, wherein said polymer is selected from the group consisting of: polyacrylates, polyacrylamides, polyvinylpyrrolidinones, polysiloxanes, polybutadienes, polyisoprenes, polyalkanes, polystyrenes, polyoxazolines or polyethers, or mixtures thereof.

13. The process of claim 12, wherein the mean molecular weight of said catalyst is 5,000 to 300,000 g/mole.

14. The process of claim 13, wherein said process is carried out in a membrane reactor.

15. The process of claims 14, wherein said process is carried out in a repetitive batch mode and said catalyst is washed between reactions.

16. The process of claim 15, wherein said catalyst is washed in such a way that no product molecules remain chemically or physically bound in the reactor.

17. The process of claim 16, wherein said process is carried out under conditions that prevent a reaction of the anhydride with the catalyst.
